(19) United States
(12) Patent Application Publication
(10) Pub. No.: US 2004/0181242 A1
(43) Pub. Date: Sep. 16, 2004

(54) Articulated suturing system

(76) Inventors: Richard S. Stack, Chapel Hill, NC (US); William L. Athas, Durham, NC (US); Dan Balbierz, Redwood City, CA (US); John Lunsford, San Carlos, CA (US)

Correspondence Address: STALLMAN & POLLOCK LLP, Suite 290, 121 Spear Street, San Francisco, CA, 94105, US

(21) Appl. No.: 10/387,157

(22) Filed: Mar. 12, 2003

(51) Int. Cl. A61B 17/10

(52) U.S. Cl. 606/139

(57) ABSTRACT

Described herein is a system for delivering a fastener to body tissue. The system includes an elongate flexible shaft extendable into a body cavity, a fastener-applying head on a distal portion of the shaft, a bending member in contact with the shaft, the bending member moveable into a first position imparting bending forces against the shaft to cause the shaft to bend. Methods for using the system are also described.

### FIELD OF THE INVENTION

The present invention relates to the field of endoscopic surgical apparatuses, and specifically to endoscopic tissue fastening apparatuses that may be articulated within the body to a target suture location.

### BACKGROUND OF THE INVENTION

Endoscopic devices are commercially available for use in applying sutures, staples, clips or other fasteners inside the body without direct visual access to the target suture location. Such devices are typically used in combination with endoscopes that allow the procedure to be observed. One example of this type of device is the SEW-RIGHT suturing device available from LSI Solutions of Victor, N.Y. This type of device is the subject of U.S. Pat. Nos. 5,431,666, 5,562,686 and 5,766,186 which are incorporated herein by reference.

Some such devices include the suture needle, or staple or clip applier, and associated components at the distal end of an inflexible straight shaft. For some applications, this configuration is not adequate because the device cannot be manipulated into target tissue locations that are well offset from the entry point of the straight shaft. Alternative suturing devices have thus come about in which the suture needle and associated components are mounted at the distal end of a flexible shaft. The flexible shaft is “piggybacked”, such as by using hook and loop-type fastening straps, onto an articulated endoscope, such that movement of the endoscope within a body cavity results in corresponding movement of the suturing device. See FIG. 1.

Unfortunately, even the piggyback configuration is substantially limited in the extent to which the device may be angled, and is insufficient to give suture access for certain applications. One such application is the implantation of devices in the proximal stomach as illustrated in FIG. 2. Implant devices for use in controlling obesity are shown and described in U.S. application Ser. No. 09/940,110, filed Aug. 27, 2001 and U.S. application Ser. No. 10/118,289 filed Apr. 8, 2002, U.S. Provisional Application No. 60/379,306 filed May 10, 2002, and U.S. application Ser. No. 10/345,666, filed Jan. 16, 2003, entitled SATIATION POUCHES AND METHODS OF USE. These applications are owned by the assignee of the present application, and the disclosures of these applications are incorporated herein by reference. One type of satiation device described in these applications is a prosthetic pouch positionable in the proximal stomach as shown in FIG. 2. The pouch 2 includes a proximal opening 4 and a smaller distal opening 6 and forms a small reservoir that collects masticated food from the esophagus—thereby limiting the amount of food that can be consumed at one time. As the pouch fills with food, it may distend, imparting pressure against the upper stomach and lower esophageal sphincter causing the patient to experience sensations of fullness. The pouch is fixed in place using clips, sutures or similar means 8 at points around the perimeter of the proximal opening 4. Wire anchor loops 9 are preferably provided for receiving sutures or clips, although the pouch could also be secured to tissue using sutures, staples, clips, etc passed directly through the pouch walls. Alternatively, windows not may be formed in the pouch for receiving sutures during attachment of the pouch to adjacent tissue. The suturing device (or clip or staple applier) used to attach the pouch in place is typically introduced through the esophagus.

Implanting devices such as satiation pouch 2 can oftentimes require attaching the pouch to the outwardly tapered region R of the proximal stomach, just below the esophagus. Depending on the anatomy of the patient receiving the implant, this procedure may require that the suture device be angled to a degree that is unattainable using the piggyback configuration of prior art suturing devices.

### SUMMARY OF THE INVENTION

Described herein is a system for delivering a fastener to body tissue. The system includes an elongate flexible shaft extendable into a body cavity, a fastener-applying head on a distal portion of the shaft, a bending member in contact with the shaft, the bending member moveable into a first position imparting bending forces against the shaft to cause the shaft to bend. Methods for using the system are also described.

### BRIEF DESCRIPTION OF THE DRAWINGS

FIG. 1 is a perspective view of a suturing device showing a prior mechanism for articulating the suture device.

FIG. 2 is a perspective view of a satiation pouch of a type that may be attached using an attachment device having the articulating features shown in FIGS. 3-8A. The pouch is shown positioned in the stomach.

FIG. 3 is a side elevation view of a first embodiment of an articulated suturing device, schematically shown positioned through an esophagus and into a stomach.

FIG. 4 is a perspective view of a slightly modified distal end for the first embodiment of FIG. 3.

FIG. 5 is a perspective view of another slightly modified distal end for the embodiment of FIG. 3.

FIG. 6A is a side elevation view of the distal end of a third embodiment of an articulated suturing device, shown in a curved position.

FIG. 6B is a side elevation view of the distal end of FIG. 6A, shown in the straight position.

FIG. 7A is a side elevation view of the distal end of a fourth embodiment of an articulated suturing device, shown in a straight position.

FIG. 7B is a side elevation view of the distal end of FIG. 7A, shown in the curved position.

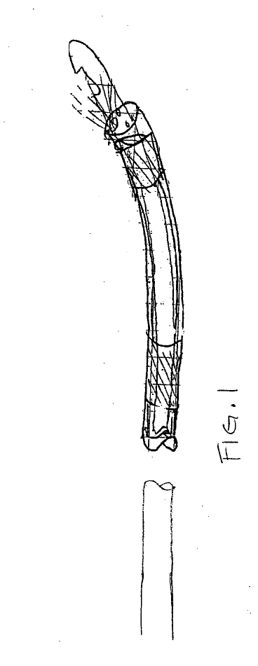

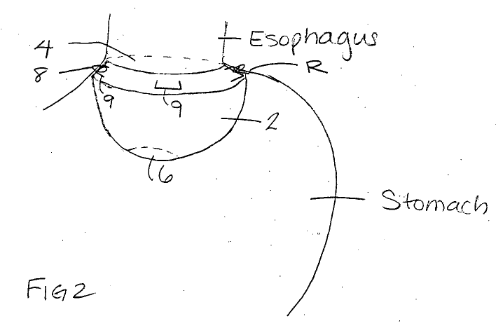

### DETAILED DESCRIPTION OF THE DRAWINGS

First Embodiment

FIG. 3 shows a first embodiment of an articulating attachment 10 attached to a suturing device 11. Suturing device 11 may be a commercially device and is preferably a type that includes a suture applier 12 at the flexible distal end of an elongate shaft 14. A suture needle (not shown) is housed within applier 12. A handle 16 is provided with a trigger 18 that, when actuated by a users hand H, causes the suture needle to pass a suture through adjacent tissue. Specific features of the firing mechanisms of the suturing device 11 are known in the art and are not described herein. It should be noted at this point that although the embodiments described in this application are described for use with suturing devices, they are equally useful with other endoscopic devices. Such endoscopic devices naturally include fastening devices for fastening implants to tissue. The term “fastening” is used herein to include using a variety of different fastening means including sutures, staples, clips, adhesives, and any other types of fastening means that may be used to connect implants to tissue.

Articulating attachment 10 includes an elongate tube 20 that extends along the exterior of shaft 14. If the articulating attachment 10 is to be provided as an “add-on” to an existing suturing device, tube 20 may be attachable to shaft 14 using a strap 21 having hook-and-loop type fasteners, or other temporary means. Alternatively, articulating attachment may be more permanently fixed to the suturing device 11 such as by permanently fixing the tube 20 to the shaft 14.

A pull-wire 22 is slidably positioned within tube 20. A distal end of the pull-wire extends from the distal end of tube 20 and is fixed to applier 12, while a proximal end of the pull-wire extends from the proximal end of tube 20 and is attached to a steering trigger 24. Pulling against steering trigger 24 in a proximal direction causes pull-wire 22 to pull suture applicator 12 into an angled position as indicated in dashed lines in FIG. 3. If articulating attachment 10 is to be a permanent attachment to suturing device 11, trigger 24 may be permanently fixed to the handle 16.

If desired, the pull-wires may take the form of ribbons that serve the added function of moving tissue aside when pulled to articulate the applier.

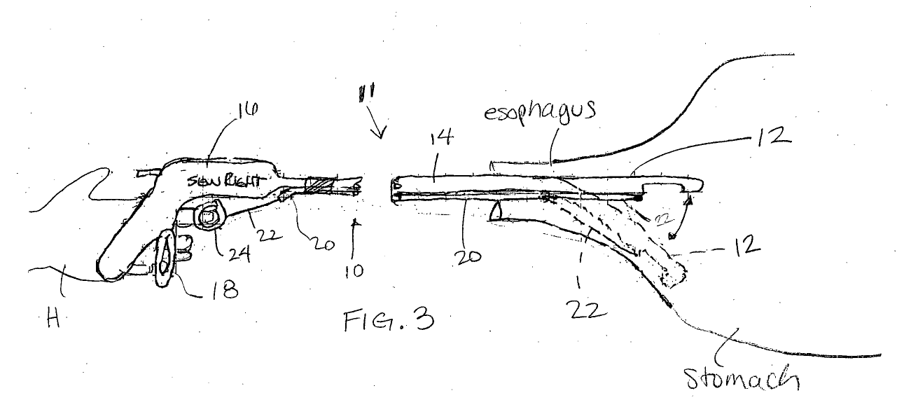

In a slightly modified embodiment shown in FIG. 4, tube 20a may be a larger-diameter tube. In this embodiment, both shaft 14a of applier head 12 and pull wire 22a extend through the tube 20a. As with the embodiment of FIG. 3, the FIG. 4 embodiment may be provided as an add-on to existing suturing devices (in which case tube 20a is telescopically positioned over shaft 14a prior to use), or as a more permanent attachment.

According to yet another variation, the pull wire may extend directly through the shaft 14 to which the applier head 12 is mounted and attached at its distal end at a location within the shaft at which bending is desired. This configuration is beneficial in that it decreases the outer diameter of the device 10.

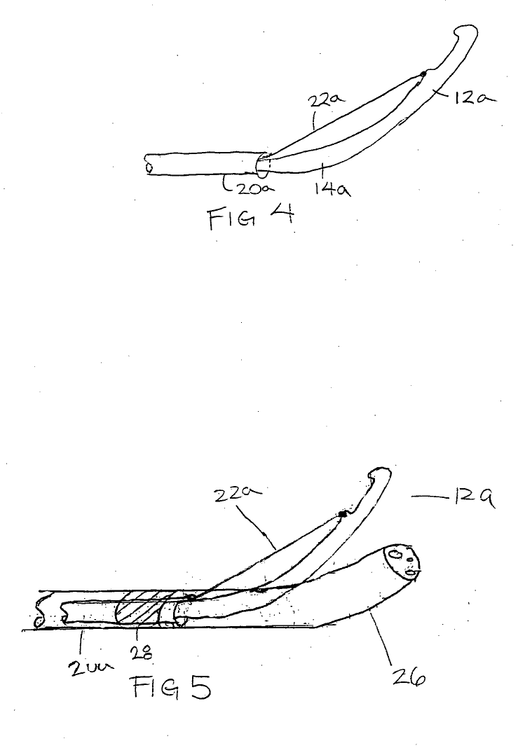

FIG. 5 shows a slight modification to the FIG. 4 embodiment, in which the tube 20a is further connected to the shaft of an endoscope 26, such as by using a strap 28 having hook-and-loop type fasteners.

It should be appreciated that additional pull wires may be attached at various points on the applier, and additional triggers may be added to pull the additional wires. Under this variation, the applier can be articulated in two or more directions.

Second Embodiment

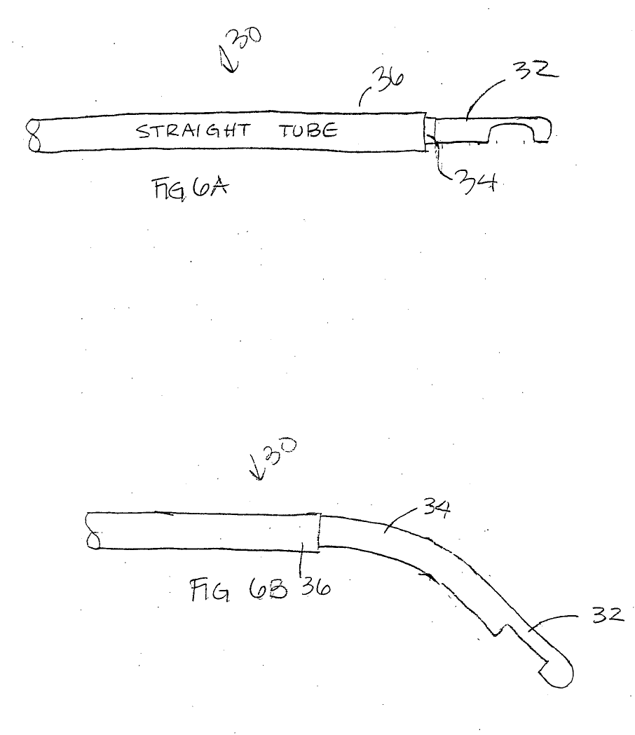

FIGS. 6A and 6B show a second embodiment of an articulated suturing device 30. In the second embodiment, suture applier 32 is coupled to a pre-curved shaft 34 as shown in FIG. 6B. Shaft 34 is formed of a material such as nitinol that can assume a straightened condition when subjected to external straightening forces, but that will resume its pre-curved shape when the external forces are removed.

Shaft is slidable within a straight cannula 36, such that the shaft 34 straightens when withdrawn into the cannula (FIG. 6A), and resumes its naturally curved shape when extended from the cannula (FIG. 6B). A trigger (which is not shown but may be similar to trigger 24 of FIG. 3) is provided for moving the cannula 36 between proximal and distal positions.

Third Embodiment

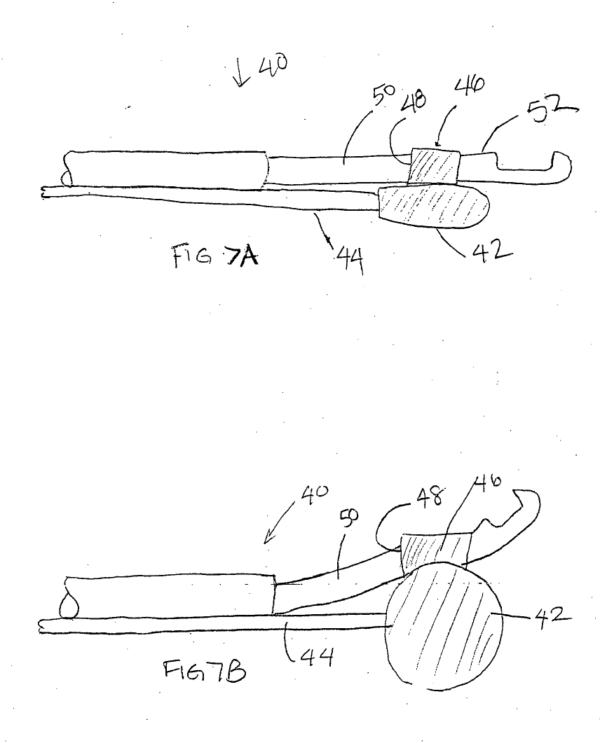

A third embodiment 40 is shown in FIGS. 7A and 7B. Embodiment 40 includes an inflatable balloon 42 mounted to a substantially straight cannula 44. Cannula is fluidly coupled to a source (not shown) of inflation medium.

A cuff 46 coupled to the balloon 42 has a central opening 48 proportioned to receive flexible shaft 50 of a suture applier 52. Inflation of the balloon causes cuff 46 to move applier 52 into an articulated position as shown in FIG. 7B. The degree of bend of the applier shaft 50 may be controlled by varying the degree to which the balloon is inflated.

As with each of the other embodiments described herein, the FIG. 7A embodiment may additionally include an endoscope for providing visualization near the operative end of the suture applier 52.

Various embodiments of articulating attachments for suturing devices have been described herein. These embodiments are given by way of example and are not intended to limit the scope of the present invention. It should be appreciated, moreover, that the various features of the embodiments that have been described may be combined in various ways to produce numerous additional embodiments. For example, each of the described embodiments may be configured as an “add-on” device to be used with existing suture devices, or provided as a more permanent feature of a suturing device.

Moreover, while various materials, shapes, implantation locations, etc. have been described for use with disclosed embodiments, others besides those disclosed may be utilized without exceeding the scope of the invention.

What is claimed is:

1. A method of attaching a medical implant in a body passage having outwardly tapered walls, comprising the steps of:
providing an elongate flexible shaft and a fastener-applying head on a distal portion of the shaft;
extending the fastener-applying head through an opening in a body and into a body passage having an outwardly tapered wall;
positioning the medical implant in the body passage;
bending the shaft to position the fastener-applying head in proximity to the wall; and
attaching the medical implant to the wall.

2. The method of claim 1 wherein the attaching step includes attaching the medical implant to the wall using a suture.

3. The method of claim 1 wherein the attaching step includes attaching the medical implant to the wall using a clip.

4. The method of claim 1 wherein the attaching step includes attaching the medical implant to the wall using a staple.

5. The method of claim 1 wherein the shaft includes a pull wire and wherein the bending step includes withdrawing the pull wire.

6. The method of claim 5 wherein the pull wire is coupled to the exterior of the shaft.

7. The method of claim 5 wherein the pull wire extends through a channel within the shaft.

8. The method of claim 1 wherein the providing step further provides an expandable member in contact with the shaft, and wherein the bending step includes expanding the expandable member to push a portion of the shaft in a lateral direction.

9. The method of claim 8 wherein the expandable member is an inflatable balloon.

10. The method of claim 1 further including the step of extending an endoscope through the body opening.

11. The method of claim 10 wherein the shaft includes a pull wire and wherein the bending step includes withdrawing the pull wire, and wherein the endoscope is independent of the pull wire.

12. The method of claim 1 wherein the bending step includes bending the shaft to an angle of up to 90 degrees.

13. The method of claim 1 wherein the method is for attaching a medical implant in a gastro-esophageal junction region in a patient having an esophagus and a stomach, wherein the outwardly-tapered wall is a wall of the gastro-esophageal junction region, and wherein the extending step includes extending the fastener-applying head through the esophagus towards the stomach.

14. A system for delivering a fastener to body tissue, comprising:
an elongate flexible shaft extendable into a body cavity;
a fastener-applying head on a distal portion of the shaft;
a bending member in contact with the shaft, the bending member moveable into a first position imparting bending forces against the shaft to cause the shaft to bend.

15. The system of claim 14 wherein the bending member is a pull wire attached to the shaft, the pull wire moveable to a retracted position to create a bend in the shaft.

16. The system of claim 15 wherein the pull wire is connected to an exterior portion of the shaft.

17. The system of claim 15 wherein the pull wire extends through an interior channel in the shaft.

18. The system of claim 14 wherein the bending member is an expandable member in contact with the shaft and expandable to impart bending forces against the shaft.

19. The system of claim 18 wherein the bending member is an inflatable balloon.

20. The system of claim 14 further comprising an endoscope extendable in the body cavity, the endoscope independent of the bending member.

21. The system of claim 14 wherein the bend is in the range of 10-90 degrees.

22. The system of claim 14 wherein the fastener-applying head is a suturing head.

23. The system of claim 14 wherein the fastener-applying head is a stapling head.

24. The system of claim 14 wherein the fastener-applying head is a clip applying head.

25. A medical implant system comprising:
a medical implant proportioned for implantation in a gastro-esophageal junction region of a body, the medical implant including a proximal opening, a distal opening, and a passage between the proximal and distal opening;
a fastener-applying device including an elongate flexible shaft extendable into a gastro-esophageal junction region, a fastener-applying head on a distal portion of the shaft, and a bending member in contact with the shaft, the bending member moveable into a first position imparting bending forces against the shaft to cause the shaft to bend.

26. The medical implant system of claim 25 wherein the medical implant is proportioned for attachment to an outwardly-tapered wall of a gastro-esophageal junction region, and wherein the bending member is moveable to impart bending forces against the shaft to cause the shaft to bend into proximity with the outwardly-tapered wall.

27. The system of claim 25 wherein the bending member is a pull wire attached to the shaft, the pull wire moveable to a retracted position to create a bend in the shaft.

28. The system of claim 27 wherein the pull wire is connected to an exterior portion of the shaft.

29. The system of claim 27 wherein the pull wire extends through an interior channel in the shaft.

30. The system of claim 25 wherein the bending member is an expandable member in contact with the shaft and expandable to impart bending forces against the shaft.

31. The system of claim 30 wherein the bending member is an inflatable balloon.

32. The system of claim 25 further comprising an endoscope extendable in the body cavity, the endoscope independent of the bending member.

33. The system of claim 25 wherein the bend is in the range of 10-90 degrees.

34. The system of claim 25 wherein the fastener-applying head is a suturing head.

35. The system of claim 25 wherein the fastener-applying head is a stapling head.

36. The system of claim 25 wherein the fastener-applying head is a clip applying head.

37. An articulating attachment attachable to a fastener-applying system having an elongate flexible shaft extendable into a body cavity and a fastener-applying head on a distal portion of the shaft, the articulating attachment comprising:
a bending member positionable in contact with the shaft, the bending member moveable into a first position imparting bending forces against the shaft to cause the shaft to bend.

38. The system of claim 37 wherein the bending member is a pull wire attachable to the shaft, the pull wire moveable to a retracted position to create a bend in the shaft.

39. The system of claim 38 further including a sleeve attached to the pull wire and positionable around a portion of the shaft.

40. The system of claim 37 wherein the bending member is an expandable member positionable in contact with the shaft and expandable to impart bending forces against the shaft.

41. The system of claim 40 wherein the bending member is an inflatable balloon.

42. The system of claim 40 further including a sleeve attached to the balloon and positionable around a portion of the shaft.
